SEQUENCE LISTING (16 sequences)

SEQ ID NO: 1 - protein, 75 aa, synthetic construct
TKCVVRFIFRGDFAWVALRAVKDHLKKEGPHWNITETNQDYMQLVVRGIHESDAKRIAKWVENRFPYARVETQCD

SEQ ID NO: 2 - protein, 75 aa, synthetic construct
TKCVVRFNFRGDMAVYALKAVKDHLKKEGPHWNITTHNDDEVYLVVRGIHESDAKRIAKWVESTIPGISVETQCD

SEQ ID NO: 3 - protein, 75 aa, synthetic construct
TKCVVRFNFRGDLATYALKAVKDHLKKEGPHWNITTHNDDEVYLVVRGIHESDAKRIAKWVESTIPGISVETQCD

SEQ ID NO: 4 - protein, 75 aa, synthetic construct
TKCVVRFIFRGDMANYALKAVKDHLKKEGPHWNITTQEEPNQYLVVRGIHESDAKRIAKWVEKRFPFAGVETQCD

SEQ ID NO: 5 - protein, 75 aa, synthetic construct
TKCVVRFVFRGDFAKYMLKAVKDHLKKEGPHWNITEGESEENQLVVRGIHESDAKRIAKWVQTRFPYIRVETQCD

SEQ ID NO: 6 - protein, 75 aa, synthetic construct
TKCVVRFIFRGDMAQYALKAVKDHLKKEGPHWNITTQEEPNQYLVVRGIHESDAKRIAKWVEKRFPFAGVETQCD

SEQ ID NO: 7 - protein, 7 aa, synthetic construct
  VARIANT 5..6: note = X can be any amino acid
  VARIANT 7: note = X is L or I
RGDLXXX

SEQ ID NO: 8 - protein, 4 aa, synthetic construct
  VARIANT 2: note = Can be A or any other amino acid
  VARIANT 3: note = Can be T or any other amino acid
  VARIANT 4: note = Can be L or I
LATL

SEQ ID NO: 9 - protein, 4 aa, synthetic construct
LATY

SEQ ID NO: 10 - protein, 4 aa, synthetic construct
LATI

SEQ ID NO: 11 - protein, 7 aa, synthetic construct
  VARIANT 5..6: note = X can be any amino acid
RGDLXXL

SEQ ID NO: 12 - protein, 7 aa, synthetic construct
RGDMAVY

SEQ ID NO: 13 - protein, 7 aa, synthetic construct
RGDLATI

SEQ ID NO: 14 - protein, 4 aa, synthetic construct
MAVY

SEQ ID NO: 15 - protein, 8 aa, synthetic construct
CSSIPYFC

SEQ ID NO: 16 - protein, 8 aa, synthetic construct
CSDYNLDC

(19) United States
(12) Patent Application Publication
(10) Pub. No.: US 2026/0297135 A1
(43) Pub. Date: Oct. 1, 2026

(54) DE NOVO DESIGNED ALPHA (V) BETA (8) INTEGRIN SELECTIVE MINIBINDER

(71) Applicants: University of Washington, Seattle, WA (US); FRED HUTCHINSON CANCER CENTER, Seattle, WA (US)

(72) Inventors: David BAKER, Seattle, WA (US); Anindya ROY, Seattle, WA (US); Hua BAI, Seattle, WA (US); John C. KRAFT II, Seattle, WA (US); Melody G CAMPBELL, Seattle, WA (US); McKaela S. SMITH, Seattle, WA (US)

(21) Appl. No.: 19/478,993

(22) PCT Filed: Jun. 12, 2024
(86) PCT No.: PCT/US2024/033514

§ 371 Date: Oct. 27, 2025

Related U.S. Application Data
(60) Provisional application No. 63/507,646, filed on Jun. 12, 2023.

(51) Int. Cl. C07K 14/00; A61K 31/404; A61K 31/439; A61K 31/44; A61K 31/4439; A61K 31/454; A61K 31/47; A61K 31/506; A61K 31/517; A61K 31/5377; A61K 38/00; A61K 39/395

(52) U.S. Cl. CPC C07K 14/00; A61K 31/404; A61K 31/439; A61K 31/44; A61K 31/4439; A61K 31/454; A61K 31/47; A61K 31/506; A61K 31/517; A61K 31/5377; A61K 39/3955; A61K 38/00

(57) ABSTRACT

Alpha(v) beta (8) integrin (αvβ8)-selective binding polypeptides and their use for treating or inhibiting cancer or tissue fibrosis are provided, where the polypeptides have an ammo acid sequence at least 50% identical to the amino acid sequence selected from SEQ ID NO: 1-6, not including any amino acid insertions, wherein the polypeptide selectively binds to αvβ8, and wherein: (a) residues 10-12 relative to the reference sequence are RGD (b) residue 13 relative to the reference sequence is F, M, or L; (c) residue 16 relative to the reference sequence is Y or V; and (d) residue 40 relative to the reference sequence is D, E, P.

A computer readable form of the Sequence Listing is filed with this application by electronic submission and is incorporated into this application by reference in its entirety. The Sequence Listing is contained in the file created on Jun. 5, 2024 having the file name “23-0157-WO_SequenceListing” and is 17,313 bytes in size.

### BACKGROUND

The RGD (Arg-Gly-Asp)-binding integrin αvβ6 and αvβ8 are clinically validated cancer and fibrosis targets of considerable therapeutic importance. Compounds that can discriminate between the two closely related integrin proteins and other RGD integrins, stabilize specific conformational states, and have sufficient stability enabling tissue restricted administration could have considerable therapeutic utility. Existing small molecules and antibody inhibitors do not have all of these properties, and hence there is a need for new approaches.

### SUMMARY

In one aspect, the disclosure provides alpha (v) beta (8) integrin (αvβ8)-selective binding polypeptides, comprising or consisting of an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from SEQ ID NO:1-6, not including any amino acid insertions, wherein the polypeptide selectively binds to αvβ8, and wherein:
(a) residues 10-12 relative to the reference sequence are RGD
(b) residue 13 relative to the reference sequence is F, M, or L;
(c) residue 16 relative to the reference sequence is Y or V; and
(d) residue 40 relative to the reference sequence is D, E, or P.

In one embodiment, residue 41 relative to the reference sequence is D, E, Y, or N. In another embodiment, residue 15 relative to the reference sequence is W, V, T, N, K, or Q. In a further embodiment, residue 16 is Y, relative to the reference sequence. In one embodiment, residue 13, relative to the reference sequence, is M or L. In another embodiment, residue 15, relative to the reference sequence, is T or V. In further embodiments, residues 13-16, relative to the reference sequence, are LATY (SEQ ID NO:9) or MAVY (SEQ ID NO:14). In other embodiments, 1, 2, 3, 4, 5, 6, 7, 8, 9, 10, 11, 12, 13, 14, or all 15 of residues 13-16, 39-42, and 63-69, relative to the reference sequence, are identical to the reference sequence. In one embodiment, the polypeptides comprise or consist of an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from SEQ ID NO:2-3, not including any amino acid insertions. In another embodiment, amino acid changes from the reference protein are conservative amino acid substitutions. In a further embodiment, amino acid substitutions relative to the reference sequence are selected from the amino acid substitutions in Table 2. In other embodiments, the polypeptides further comprise one or more additional functional domains added at the N-terminus, the C-terminus of the polypeptide, and/or one or more insertion sites selected from residues 28-31, 38, 48, and 50-52 relative to the reference sequence.

The disclosure also provides nucleic acids encoding the polypeptide of any embodiment herein, expression vectors comprising the nucleic acid operatively linked to a control sequence, such as a promoter, host cells comprising the nucleic acid and/or the expression vector of any embodiment, recombinant cells expressing the polypeptide of any embodiment, and pharmaceutical compositions, comprising the polypeptide, nucleic acid, expression vector, host cell, or recombinant cell of any embodiment; and a pharmaceutically acceptable carrier.

In another aspect, the disclosure provides methods for treating or inhibiting an αvβ8 (+) tumor, comprising administering to a subject in need thereof an amount of the polypeptide, nucleic acid, expression vector, host cell, and/or pharmaceutical composition of any embodiment herein effective to treat or inhibit the tumor in the subject. In one embodiment, the method further comprises administering to the subject one or more other therapeutic agents selected from the group consisting of radiation therapy, an angiogenesis inhibitor, a protein kinase inhibitor, a proteasome inhibitor, an immune checkpoint inhibitor, an mTOR inhibitor, a PI3K inhibitor, a histone deacetylase inhibitor, and a Hedgehog pathway blocker. In a further embodiment, the tumor is selected from the group consisting of a colorectal tumor, a glioblastoma, and a melanoma.

The disclosure also provides methods for detecting an αvβ8 (+) tumor or immune cell population, comprising administering to a subject suspected of having an αvβ8 (+) tumor or immune cell population an amount of the polypeptide of any embodiment herein effective to detect the tumor in the subject.

In another aspect, the disclosure provides methods for treating or inhibiting tissue fibrosis, comprising administering to a subject in need thereof an amount of the polypeptide, nucleic acid, expression vector, host cell, and/or pharmaceutical composition of any embodiments herein effective to treat or inhibit the tissue fibrosis in the subject. In one embodiment, the tissue fibrosis is selected from the group consisting of pulmonary fibrosis, liver fibrosis, and renal fibrosis.

### DESCRIPTION OF THE FIGURES

FIG. 1. Computational design of αvβ8 selective minibinders. a) Crystal structure of β8 (PDB ID 60M2) overlaid on the structure of αvβ6 integrin in complex with the L-TGF-β3 peptide RGDLXX (L/I) (SEQ ID NO: 7)(PDB ID 4UM9). b) Polar interactions between the RGD motif and αvβ8 integrin (PDB ID 60M2). c) Hydrophobic packing of LXX(L/I) (SEQ ID NO: 8) motif of L-TGF-β3 peptide with SDL2 of αvβ6 (PDB ID 4UM9). Leu247 packs optimally against Y185 from SDL2 of β6. d) Hydrophobic packing of LXX(L/I) (SEQ ID NO: 8) of L-TGF-β1 peptide with SDL2 of αvβ8 (PDB ID 60M2). I221 of L-TGF-β1 peptide packs less tightly against L174 on SDL2 of β8 compared to the homologous interactions in panel d. e) Charge reversal on β subunit: β8 contains K304 whereas the equivalent position on β6 is E316. f) Small α/β ferredoxin folds were able to scaffold the RGDLXX(L/I) (SEQ ID NO: 7) binding loop without clashing with the integrin while making additional contacts with both α and β subunit (α- and β-loop respectively) g) Loop extension strategy to design an αvβ8 selective minibinder: to make more extensive contacts to the β8 subunit the β-loop was resampled by one residue insertion (surface representation for β8 subunit, PDB ID 6OM2). In addition to the loop extension, the LXX (L/I) (SEQ ID NO: 8) motif was redesigned using Rosetta. h) Partial sequence alignment of SDL2 of the β6/β8 subunits is shown highlighting two key positions packing against the LXX(L/I) (SEQ ID NO: 8) motif of the L-TGF-β ligand (1183 and Y185 in SLD2-β6, and Y172 and L174 in SDL2-β8) (top to bottom SEQ ID NOs: 15-16).

FIG. 2. Competition assay using yeast surface display technique to determine selectivity of designed αvβ8 binders. a) Designs were expressed using yeast surface display technique and incubated with 50 nM of FLAG-tagged αvβ8 (to confirm binding to αvβ8) and in the presence or absence of 625 nM of unlabelled αvβ6 (the purpose of this unlabeled αvβ6 integrin is to compete away non-αvβ8-selective binders that also bind to αvβ6). This assay setup allows for identification of αvβ8-selective binders, which are verified only if a binding population is present in the top right quadrant in both the “no_αvβ6” and “625 nM αvβ6” conditions (the only construct in which two clear binding populations are present in the top right quadrant is abv8_#3 [MAVY]). LATI is the amino acid sequence of the residues that immediately follow the RGD motif in endogenous human L-TGFβ (latent TGFβ). Binding was detected by anti-FLAG-PE conjugated antibody from Abcam (Product #ab72469). Based on these data, avb8 #3 was identified as a lead candidate that exhibited exquisite selectivity for integrin αvβ8. b) Zoomed in data of bold box in FIG. 2A. Effect of RGDLXXL (SEQ ID NO: 11) motif on selectivity of the designed binders. The-RGDMAVY (SEQ ID NO: 12) motif, present in avb8_#3, also referred to as B8_BP_dslf; (SEQ ID NO:2) selectively binds to αvβ8 as shown by the two populations in the top right quadrant (left panel): one population is in the presence of 625 nM αvβ6 and the other population has no αvβ6 present to compete for binding. Mutation of the RGDMAVY (SEQ ID NO: 12) motif to RGDLATI (SEQ ID NO: 13) (present in avb8_#12) completely abrogates selectivity towards αvβ8. B8_BP_dslf (SEQ ID NO:2) is highly selective to αvβ8. αvβ8-selective B8_BP_dslf does not bind to any other RGD binding integrins as confirmed by BLI (FIG. 3a).

FIG. 3. Binding selectivity studies. a) Binding for the B8_BP_dslf and its point mutants against αvβ6 and αvβ8 and selectivity for B8_BP_dslf for RGD integrins. b) Binding affinities (Kd) of B8_BP_dslf (SEQ ID NO:2) point mutants in the MAVY (SEQ ID NO: 14) motif (residues 13-16 in SEQ ID NO:2) to integrins αvβ6 and αvβ8, determined by BLI, and binding affinities (Kd) and fold-selectivity values of B8_BP-LATY (SEQ ID NO:3; the LATY motif is present at residues 13-16 in SEQ ID NO: 9) to all eight RGD integrins.

FIG. 4. Binding selectivity studies. a, b) Competitive inhibition of h-LAP₁ binding to (a) αvβ6 and (b) αvβ8 by designed inhibitors and control small molecule PLN-74809. c) Heatmap of IC₅₀ values for h-LAP₁ binding assays in a and b.

FIG. 5. Structural characterization. a) Representative 2D class averages of integrin with and without minibinder. For αvβ8, no open headpieces were observed, with or without minibinder. b) CryoEM density map of αvβ8 bound to minibinder B8_BP_dslf. B8_BP_dslf binds the integrin ligand binding cleft between the av and β8 subunits, however the conformation of αvβ8 remains in the closed headpiece conformation. The sharpened, locally refined cryoEM map is shown, superimposed with the unsharpened map showing all domains in the αvβ8 ectodomain construct in semi-transparent white. c, d) An overlay of the designed αvβ8+B8_BP_dslf model (gray) and the experimentally determined model. Although the overall angle of the minibinder is shifted, the RGD loop positioning is as predicted. Insets in c) and d) are magnified in panels e) and f), respectively. e) E41 from β-loop makes backbone level hydrogen bond with 1216 from β8 subunit and D40 makes salt bridge interaction with K304 from β8 subunit (panel i). f) Experimental vs designed (gray) packing pattern of the MAVY (SEQ ID NO: 14) motif and SDL2 of αvβ8.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2nd Ed. (R. I. Freshney. 1987. Liss, Inc. New York, NY), Gene Transfer and Expression Protocols, pp. 109-128, ed. E. J. Murray, The Humana Press Inc., Clifton, N.J.), RosettaCommons.org, and the Ambion 1998 Catalog (Ambion, Austin, TX).

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

In all embodiments of polypeptides disclosed herein, any N-terminal methionine residues are optional (i.e.: the N-terminal methionine residue may be present or may be deleted).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application

In one aspect, the disclosure provides alpha (v) beta (8) integrin (αvβ8)-selective binding polypeptides comprising or consisting of an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from SEQ ID NO:1-6, not including any amino acid insertions, wherein the polypeptide selectively binds to αvβ8, and wherein:
(a) residues 10-12 relative to the reference sequence are RGD
(b) residue 13 relative to the reference sequence is F, M, or L;
(c) residue 16 relative to the reference sequence is Y or V; and
(d) residue 40 relative to the reference sequence is D, E, or P.

The polypeptides disclosed here are hyperstable RGD-containing miniproteins that are highly selective for the human αvβ8 integrin, such that they bind with a picomolar affinity (K_{d}) to the human αvβ8 integrin that is at least 1,000-fold higher affinity than the affinity at which they bind to the other human RGD integrins (e.g., ανβ1, ανβ3, ανβ5, ανβ6, α5β1, α8ß1, αIIbβ3), as determined by biolayer interferometry in which protein binders are immobilized on Ni-NTA and streptavidin sensor tips. The tips were then dipped into wells containing different concentrations of integrin, and association and dissociation steps were recorded for 900 s and 1200 s respectively.

The amino acid sequence of SEQ ID NO: 1-6 are shown in Table 1. The proteins comprise a rigid backbone formation of an RGD loop-beta sheet-alpha helical domains connected by amino acid linkers. Within SEQ ID NO:1-6, the RGD-loop domain (binds to the ligand binding site at the interface between the alpha and beta chains) comprises residues 10 to 16, the beta-loop domain (binds to the beta chain on the integrin) comprises residues 39 to 42, and the alpha-loop domain (binds to the alpha chain on the integrin) comprises residues 63 to 69. In one embodiment, 1, 2, 3, 4, 5, 6, 7, 8, 9, 10, 11, 12, 13, 14, or all 15 of residues 13-16, 39-42, and 63-69, relative to the reference sequence, are identical to the reference sequence. These are the interface residues, in addition to residues 10-12

In one embodiment, the polypeptides comprise or consist of an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from SEQ ID NO: 2-3, not including any amino acid insertions, wherein the polypeptide selectively binds to αvβ8. In various embodiments, the polypeptides comprise an amino acid sequence at least 60% identical to the amino acid sequence selected from SEQ ID NO:1-6 or SEQ ID NO:2-3; or at least 70% identical to the amino acid sequence selected from SEQ ID NO:1-6 or SEQ ID NO: 2-3; or at least 80% identical to the amino acid sequence selected from SEQ ID NO:1-6 or SEQ ID NO:2-3; or at least 90% identical to the amino acid sequence selected from SEQ ID NO: 1-6 or SEQ ID NO:2-3; or at least 95% identical to the amino acid sequence selected from SEQ ID NO:1-6 or SEQ ID NO:2-3; in all embodiments not including any insertions, wherein residues 10-12 relative to the reference polypeptide are RGD, and wherein the polypeptide selectively binds to integrin αvβ8. In other embodiments, the polypeptides consist of an amino acid sequence at least 60% identical to the amino acid sequence selected from SEQ ID NO:1-6 or SEQ ID NO:2-3; or at least 70% identical to the amino acid sequence selected from SEQ ID NO:1-6 or SEQ ID NO:2-3; or at least 80% identical to the amino acid sequence selected from SEQ ID NO:1-6 or SEQ ID NO:2-3; or at least 90% identical to the amino acid sequence selected from SEQ ID NO:1-6 or SEQ ID NO:2-3; or at least 95% identical to the amino acid sequence selected from SEQ ID NO:1-6 or SEQ ID NO: 2-3; in all embodiments not including any insertions, wherein residues 10-12 relative to the reference polypeptide are RGD, and wherein the polypeptide selectively binds to integrin ανβ8.

One or more amino acid insertions can be made at residues 28-31, 38, 48, and 50-52 without affecting αvβ8 binding. These residues are referred to as “insertion sites”. Insertions are permissible at these regions because they are (1) flexible loop regions that can accommodate residue insertions without altering the rigid backbone conformation of beta sheet and alpha helix secondary structures and (2) they are not involved in interactions with the αvβ8 integrin. Amino acid insertions may be single residues, multiple residues, or functional domains. In one embodiment, an amino acid insertion is present, but does not result in elimination of any residues in the polypeptide. In another embodiment, an amino acid insertion is present, and does result in elimination of 1, 2, 3, or 4 contiguous insertion sites.

The position of residues in the polypeptides of the disclosure are “relative to” the position of residues in the reference sequence; this does not necessarily mean that the residue number in the polypeptide of the disclosure will be identical to the residue number in the reference sequence. For example, residues 10-12 of the reference sequences of SEQ ID NO: 1-6 are RGD. Polypeptides of the disclosure have an RGD tripeptide that is at a position corresponding to residues 10-12 in the reference sequence, but not necessarily at residues 10-12 in the polypeptides of the disclosure. Those of skill in the art will understand that the polypeptides of the disclosure may be fused to other functional domains, including N-terminal domains, such that the RGD residues in the polypeptides will not be at positions 10-12, but the polypeptides will still have the RGD triad at residues 10-12 relative to the reference polypeptide selected from SEQ ID NO:1-14. Similarly, the polypeptides of the disclosure may include insertions at residues 28-31, 38, 48, and 50-52 relative to the reference sequence, as noted above.

TABLE 1

| | | αvβ8 | | |
|---|---|---|---|---|
| SEQ ID | | Binding | Rel. | |
| NO | Name | affinity | selecivity | Amino acid sequence |
| 1 | avb8_2 | + | + | TKCVVRFIFRGDFAWVALRAVKDHLKKEGPHWNITETNQDY |
| | | | | MQLVVRGIHESDAKRIAKWVENRFPYARVETQCD |
| 2 | avb8_3 | ++++ | ++++ | TKCVVRFNFRGDMAVYALKAVKDHLKKEGPHWNITTHNDDE |
| | (B8_BP_dslf) | | | VYLVVRGIHESDAKRIAKWVESTIPGISVETQCD |
| 3 | avb8_3_LATY | ++++ | ++++ | TKCVVRFNFRGDLATYALKAVKDHLKKEGPHWNITTHNDDE |
| | | | | VYLVVRGIHESDAKRIAKWVESTIPGISVETQCD |
| 4 | avb8_5 | + | ++ | TKCVVRFIFRGDMANYALKAVKDHLKKEGPHWNITTQEEPN |
| | | | | QYLVVRGIHESDAKRIAKWVEKRFPFAGVETQCD |
| 5 | avb8_6 | ++ | ++ | TKCVVRFVFRGDFAKYMLKAVKDHLKKEGPHWNITEGESEE |
| | | | | NQLVVRGIHESDAKRIAKWVQTRFPYIRVETQCD |
| 6 | avb8_9 | ++ | ++ | TKCVVRFIFRGDMAQYALKAVKDHLKKEGPHWNITTQEEPN |
| | | | | QYLVVRGIHESDAKRIAKWVEKRFPFAGVETQCD |

In one embodiment, residue 41 relative to the reference sequence is D, E, Y, or N. Residue 41 is present in the β-loop and makes a backbone level hydrogen bond with I216 from the β8 subunit.

In another embodiment, residue 15 relative to the reference sequence is W, V, T, N, K, or Q. In another embodiment, residue 15 relative to the reference sequence is T or V, which are smaller residues that allow optimal packing of when residues 15 is Y, and improvesαvβ8 selectivity.

In a further embodiment, residue 16, relative to the reference sequence, is Y. Residue 16 was shown to be important for binding affinity and selectivity for αvβ8, as Y16 forms stabilizing interactions with A115 of the 88 subunit and interacts with the less bulky L174 in the β8-SDL2 loop.

In various further embodiments, one of the following is true:
(a) relative to the reference sequence, residue 13 is F, residue 15 is W, residue 16 is V, residue 40 is D, and residue 41 is Y;
(b) relative to the reference sequence, residue 13 is M, residue 15 is V, residue 16 is Y, residue 40 is D, and residue 41 is E;
(c) relative to the reference sequence, residue 13 is L, residue 15 is T, residue 16 is Y, residue 40 is D, and residue 41 is E;
(d) relative to the reference sequence, residue 13 is M, residue 15 is N, residue 16 is Y, residue 40 is P, and residue 41 is N;
(e) relative to the reference sequence, residue 13 is F, residue 15 is K, residue 16 is Y, residue 40 is E, and residue 41 is E; or
(f) relative to the reference sequence, residue 13 is M, residue 15 is Q, residue 16 is Y, residue 40 is P, and residue 41 is N.

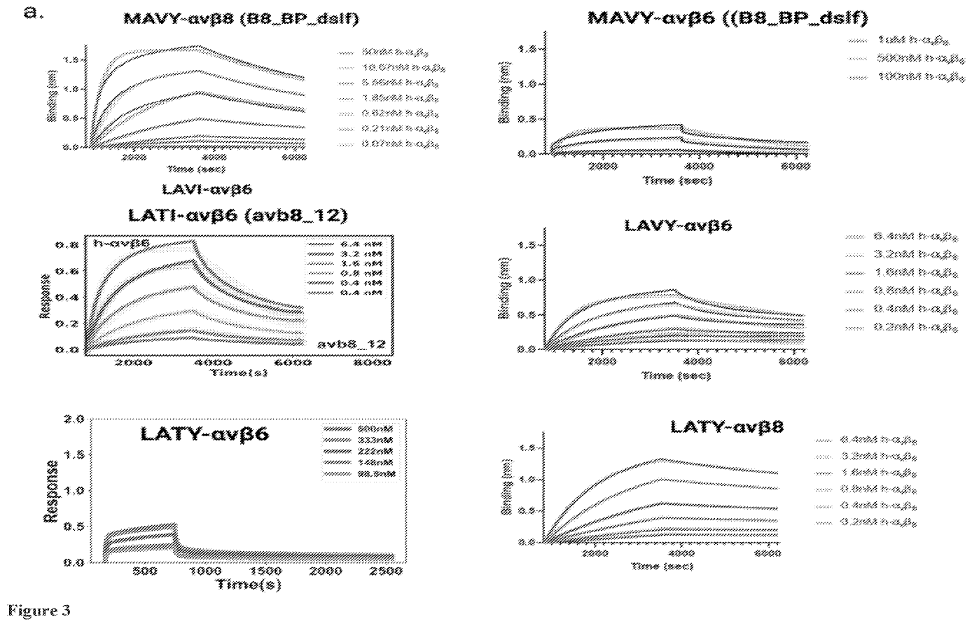

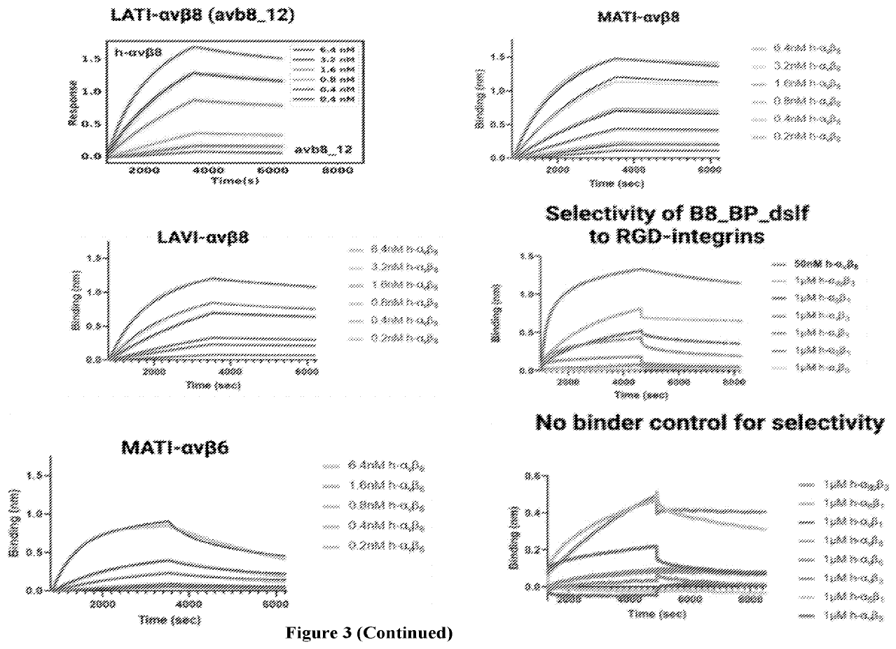

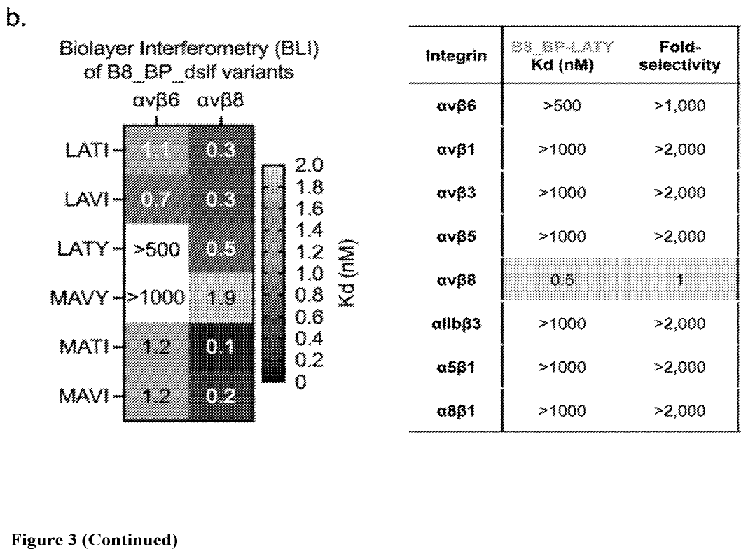

In one embodiment, residue 13, relative to the reference sequence, is Met (M) or Leu (L). Binders residues 13-16 identified as LATY and MAVY were optimally selective for αvβ8 (FIG. 3B).

Residue 40 was shown to form salt bridge interaction with K304 of the β8 subunit.

In specific embodiments, one of the following is true:
(a) relative to the reference sequence, residue 13 is M, residue 15 is V, residue 16 is Y, residue 40 is D, and residue 41 is E; or
(b) relative to the reference sequence, residue 13 is L, residue 15 is T, residue 16 is Y, residue 40 is D, and residue 41 is E.

In one embodiment, amino acid substitutions relative to the reference sequence are selected from those disclosed in Table 2. Potential substitutions represent shared chemical properties of the amino acids. Terms used in Table 2 have the following meaning
Small Polar or hydrophobic: S, T, Q, N, V, D, or E;
Hydrophobic: A, V, L, I, F, Y, W, M, P, or G;
Polar (Charged or Neutral): S, T, Q, N, D, E, K, R, or Y;
Polar (Negatively charged or Neutral): S, T, Q, N, D, E, or Y;
Polar (Positively charged or Neutral); S, T, Q, N, K, R, or Y;
Small Polar: S, T, Q, or D;
Affinity, Hot spot: Mutation causes loss of binding;
Affinity, Selectivity: Contributes to affinity and selectivity;
Affinity: Contributes to affinity; and
Insertion: amino acid insertions may be incorporated at the identified position.

TABLE 2

| Position | Identity | Function | May be substituted with |
|---|---|---|---|
| 1 | T | Structural | small polar or hydrophobic |
| 2 | K | Structural | Polar (charged or neutral) |
| 3 | C | Structural, | Hydrophobic |
| | | disulfide | |
| 4 | V | Structural | Hydrophobic |
| 5 | V | Structural | Hydrophobic |
| 6 | R | Structural | Polar |
| 7 | F | Structural | Hydrophobic |
| 8 | N | Structural | Polar |
| 9 | F | Structural | Hydrophobic |
| 10 | R | Affinity, Hot spot | N/A |
| | | Interface residue | |
| 11 | G | Affinity, Hot spot | N/A |
| | | Interface residue | |
| 12 | D | Affinity, Hot spot | N/A |
| | | Interface residue | |
| 13 | M | Affinity, | Hydrophobic, specifically F, |
| | | Selectivity | M, or L |
| | | Interface residue | |
| 14 | A | Structural | small polar or hydrophobic |
| | | Interface residue | |
| 15 | V | Structural | polar or hydrophobic, |
| | | Interface residue | specifically W, V, T, N, K, or |
| | | | Q |
| 16 | Y | Affinity, | V |
| | | Selectivity | |
| | | Interface residue | |
| 17 | A | Structural | small hydrophobic |
| 18 | L | Structural | Hydrophobic |
| 19 | K | Structural | Polar |
| 20 | A | Structural | hydrophobic |
| 21 | V | Structural | Hydrophobic |
| 22 | K | Structural | Polar (charged or neutral) |
| 23 | D | Structural | Polar (neutral or negatively |
| | | | charged) |
| 24 | H | Structural | Polar (charged or neutral) |
| 25 | L | Structural | Hydrophobic |
| 26 | K | Structural | Polar (charged or neutral) |
| 27 | K | Structural | Polar (charged or neutral) |
| 28 | E | Structural | Polar (negatively charged or |
| | | | neutral) |
| | | | Insertion |
| 29 | G | Structural | small polar or hydrophobic |
| | | | Insertion |
| 30 | P | Structural | small polar or hydrophobic |
| | | | Insertion |
| 31 | H | Structural | Polar (charged or neutral) |
| | | | Insertion |
| 32 | W | Structural | Hydrophobic |
| 33 | N | Structural | Polar (charged or neutral) |
| 34 | I | Structural | Hydrophobic |
| 35 | T | Structural | polar or hydrophobic |
| 36 | T | Structural | polar or hydrophobic |
| 37 | H | Structural | polar or hydrophobic |
| 38 | N | Structural | Polar (charged or neutral) |
| | | | Insertion |
| 39 | D | Structural | Polar (charged or neutral) |
| | | Interface residue | |
| 40 | D | Affinity | Polar (Negatively charged or |
| | | Interface residue | neutral), specifically D, E, |
| | | | or P |
| 41 | E | Affinity | Polar (Negatively or neutral), |
| | | Interface residue | specifically D, E, Y, or N |
| 42 | V | Structural | Hydrophobic |
| | | Interface residue | |
| 43 | Y | Structural | Hydrophobic |
| 44 | L | Structural | Hydrophobic |
| 45 | V | Structural | Hydrophobic |
| 46 | V | Structural | Hydrophobic |
| 47 | R | Structural | Polar (charged or neutral) |
| 48 | G | Structural | P, or small polar |
| | | | Insertion |
| 49 | I | Structural | Hydrophobic |
| 50 | H | Structural | Polar (charged or neutral) |
| | | | Insertion |
| 51 | E | Structural | Polar (charged or neutral) |
| | | | Insertion |
| 52 | S | Structural | Polar |
| | | | Insertion |
| 53 | D | Structural | Polar (charged or neutral) |
| 54 | A | Structural | small polar or hydrophobic |
| 55 | K | Structural | Polar (charged or neutral) |
| 56 | R | Structural | Polar (charged or neutral) |
| 57 | I | Structural | Hydrophobic |
| 58 | A | Structural | Hydrophobic |
| 59 | K | Structural | Polar (charged or neutral) |
| 60 | W | Structural | Hydrophobic |
| 61 | V | Structural | small hydrophobic |
| 62 | E | Structural | Polar (charged or neutral) |
| 63 | S | Structural | Small polar or hydrophobic |
| | | Interface residue | |
| 64 | T | Structural | Polar |
| | | Interface residue | |
| 65 | I | Structural | Hydrophobic |
| | | Interface residue | |
| 66 | P | Structural | Glycine, small polar |
| | | Interface residue | |
| 67 | G | Structural | Proline, small polar |
| | | Interface residue | |
| 68 | I | Structural | Hydrophobic |
| | | Interface residue | |
| 69 | S | Structural | small polar or hydrophobic |
| | | Interface residue | |
| 70 | V | Structural | Hydrophobic |
| 71 | E | Structural | Polar (charged or neutral) |
| 72 | T | Structural | small polar or hydrophobic |
| 73 | Q | Structural | Polar (charged or neutral) |
| 74 | C | Structural, | Hydrophobic |
| | | disulfide | |
| 75 | D | Structural | Polar (charged or neutral) |

In one embodiment of each of the above aspects, amino acid substitutions relative to the reference peptide domains are conservative amino acid substitutions. As used herein, “conservative amino acid substitution” means a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gin and Asn). Other such conservative substitutions, e.g., substitutions of entire regions having similar hydrophobicity characteristics, are known. Polypeptides comprising conservative amino acid substitutions can be tested in any one of the assays described herein to confirm that a desired activity, e.g. antigen-binding activity and specificity of a native or reference polypeptide is retained. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser(S), Thr (T), Cys (C), Tyr (Y), Asn (N), Gin (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In a further embodiment, the polypeptides may comprise an insertion in one or more loop regions of the polypeptide (i.e., residues 28-31, 38, 48, and 50-52). The insertion may be any one or more amino acid, and may comprise a functional domain as described in the next paragraph, or one or more amino acids for additional spacing or for any other purpose. In one embodiment, an insertion in the loop regions is 1-3, 1-2, 1, 2, or 3 amino acids in length. In certain embodiments, amino acids or amino acid domains (such as a functional domain) may be inserted in the loop region. The polypeptides of the disclosure may include any such insertion, and in these embodiments the polypeptide would still comprise the reference amino acid sequence, with an interruption at the site of insertion.

In another embodiment of any of the above aspects, the polypeptide further comprises one or more additional functional domains added at the N-terminus and/or the C-terminus of the polypeptide, and/or one or more insertion site (i.e., residues 28-31, 38, 48, and 50-52). Any suitable functional domain(s) may be added as suitable for an intended purpose, including but not limited to albumin (to improve serum half-life), Fc domains, a receptor targeting domain, therapeutics, diagnostics, molecular probes such as fluorescent proteins, a tag (including but not limited to a polyhistidine tag), etc.

In another embodiment, the polypeptide binds to the human αvβ8 integrin with a picomolar affinity (K_{d}) that is at least 1,000-fold higher than the affinity at which the polypeptide binds to any one of another human RGD integrins selected from the group consisting of αvβ1, αvβ3, αvβ5, αvβ6, α5β1, α8β1, and αIIbβ3

In a further aspect, the present disclosure provides nucleic acids, including isolated nucleic acids, encoding the polypeptides of the present disclosure. The isolated nucleic acid sequence may comprise RNA or DNA. Such isolated nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the invention.

In another aspect, the present disclosure provides expression vectors comprising the nucleic acid of any aspect of the invention operatively linked to a suitable control sequence. “Expression vector” includes vectors that operatively link a nucleic acid coding region or gene to any control sequences capable of effecting expression of the gene product. “Control sequences” operably linked to the nucleic acid sequences of the invention are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors include but are not limited to, plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector (including but not limited to a retroviral vector or oncolytic virus), or any other suitable expression vector. In some embodiments, the expression vector can be administered in the methods of the disclosure to express the polypeptides in vivo for therapeutic benefit.

In a further aspect, the present disclosure provides host cells that comprise the expression vectors, polypeptides, and/or nucleic acids disclosed herein, wherein the host cells can be either prokaryotic or eukaryotic. The cells can be transiently or stably engineered to incorporate the expression vector of the invention, using techniques including but not limited to bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press); Culture of Animal Cells: A Manual of Basic Technique, 2^{nd} Ed. (R. I. Freshney. 1987. Liss, Inc. New York, NY)). A method of producing a polypeptide according to the invention is an additional part of the invention. The method comprises the steps of (a) culturing a host according to this aspect of the invention under conditions conducive to the expression of the polypeptide, and (b) optionally, recovering the expressed polypeptide. The expressed polypeptide can be recovered from the cell free extract, but preferably they are recovered from the culture medium.

In another embodiment, the disclosure provides pharmaceutical compositions, comprising:
(a) the polypeptide, nucleic acid, expression vector, host cell, or recombinant cell of any preceding claims; and
(b) a pharmaceutically acceptable carrier.

In certain embodiments, the carrier in a pharmaceutical composition can be either aqueous or non-aqueous in nature. For example, in certain embodiments, a suitable vehicle or carrier can be water for injection, physiological saline solution or artificial cerebrospinal fluid, possibly supplemented with other materials common in compositions for parenteral administration. In certain embodiments, the saline comprises isotonic phosphate-buffered saline. In certain embodiments, neutral buffered saline or saline mixed with serum albumin are further exemplary vehicles. In certain embodiments, pharmaceutical compositions comprise Tris buffer of about pH 7.0-8.5, or acetate buffer of about pH 4.0-5.5, which can further include sorbitol or a suitable substitute therefore. In certain embodiments, a composition comprising an immunomodulatory fusion protein is prepared for storage by mixing the selected composition having the desired degree of purity with optional formulation agents (Remington's Pharmaceutical Sciences, supra) in the form of a lyophilized cake or an aqueous solution. Further, in certain embodiments, a composition comprising an immunomodulatory fusion protein is formulated as a lyophilizate using appropriate excipients such as sucrose.

The compositions may be used, for example, in the methods disclosed herein. The compositions may further comprise for example, (a) a lyoprotectant; (b) a surfactant; (c) a bulking agent; (d) a tonicity adjusting agent; (e) a stabilizer; (f) a preservative and/or (g) a buffer. In some embodiments, the buffer in the pharmaceutical composition is a Tris buffer, a histidine buffer, a phosphate buffer, a citrate buffer or an acetate buffer. The composition may also include a lyoprotectant, e.g. sucrose, sorbitol or trehalose. In certain embodiments, the composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the composition includes a bulking agent, like glycine. In yet other embodiments, the composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the composition additionally includes a stabilizer, e.g., a molecule which substantially prevents or reduces chemical and/or physical instability of the nanostructure, in lyophilized or liquid form. Exemplary stabilizers include sucrose, sorbitol, glycine, inositol, sodium chloride, methionine, arginine, and arginine hydrochloride.

The polypeptide, fusion protein, nucleic acid, expression vector, and/or host cell may be the sole active agent in the composition, or the composition may further comprise one or more other agents suitable for an intended use. In various non-limiting embodiments, such other agents may include angiogenesis inhibitors (including but not limited to axitinib, bevacizumab, cabozantinib, everolimus, lenalidomide, lenvatinib mesylate, pazopanib, ramucirumab, regorafenib, sorafenib, sunitinib, thalidomide, vandetanib, and ziv-aflibercept), immune checkpoint inhibitors (including, but not limited to, pembrolizumab, nivolumab, and cemiplimab as anti-PD-1 antibodies, ipilimumab as an anti-CTLA-4 antibody, and atezolizumab, avelumab, and durvalumab as anti-PD-L1 antibodies), and other cancer growth inhibitors including but not limited to tyrosine kinase inhibitors (including but not limited to alectinib, brigatinib, ceritinib, crizotinib, entrectinib, lorlatinib, ALK, I, bosutinib, dasatinib, imatinib, nilotinib, ponatinib, afatinib, dacomitinib, erlotinib, gefitinib, lapatinib, neratinib, osimertinib, vandetanib, gilteritinib, midostaurin, erdafitinib, ruxolitinib, larotrectinib, axitinib, carbozantinib, lenvatinib, pazopanib, regorafenib, sorafenib, sunitinib, dabrafenib, encorafenib, vemurafenib, acalabrutinib, ibrutinib, binimetinib, cobimetinib, trametinib, abemaciclib, palbociclib, or ribociclib), proteasome inhibitors (including but not limited to ortezomib, carfizomib, ixazomib, delanzomib, oprozomib, and marizomib), mTOR inhibitors (including but not limited to everolimus, sirolimus, temsirolimus, everolimus, sirolimus, sirolimus protein-bound, and everolimus), PI3K inhibitors (including but not limited to copanlisib, alpelisib, idelalisib, duvelisib and umbralisib), histone deacetylase inhibitors (including but not limited to vorinostat, romidepsin, panobinostat, and belinostat), and Hedgehog pathway blockers (including but not limited to vismodegib, sonidegib, and glasdegib).

The polypeptide, nucleic acid, expression vector, host cell, recombinant cell, or pharmaceutical composition of any embodiment may be used for various purposes, including but not limited to treating and/or detecting αvβ8 (+) tumors or immune cells in vivo; blocking αvβ8-mediated TGF-B signaling in vitro or in vivo; use in combination immuno-oncology therapeutic regimens that include immune checkpoint blockade (e.g., anti-PD1 or anti-PD-L1), chemotherapy, and/or radiotherapy; and treating pulmonary fibrosis such as Idiopathic Pulmonary Fibrosis (IPF) or any other fibrotic tissue pathology such as in cancer or inflammatory and autoimmune diseases.

In one embodiment, the disclosure provides methods for treating or inhibiting an αvβ8 (+) tumor, comprising administering to a subject in need thereof an amount of the polypeptide, nucleic acid, expression vector, host cell, and/or pharmaceutical composition of any embodiment or combination of embodiments disclosed herein effective to treat or inhibit the tumor in the subject. The methods can be used to treat any suitable tumor, including but not limited to colorectal cancer, glioblastoma, and melanoma. See, for example, Liu et al., Pharmacology and Therapeutics, Volume 247, July 2023, 108458, which is incorporated herein by reference in its entirety.

The polypeptides, fusion proteins, nucleic acids, expression vectors, and/or host cells made be administered as the sole therapeutic agent, or may be administered together with (i.e.: combined or separately) one or more other therapeutic agents, including but not limited to tumor resection, chemotherapy, radiation therapy, and immunotherapy (such as checkpoint inhibitors). In various non-limiting embodiments, other agents that may be administered in the methods of the disclosure for treating cancer include angiogenesis inhibitors (including but not limited to axitinib, bevacizumab, cabozantinib, everolimus, lenalidomide, lenvatinib mesylate, pazopanib, ramucirumab, regorafenib, sorafenib, sunitinib, thalidomide, vandetanib, and ziv-aflibercept), immune checkpoint inhibitors (including, but not limited to, pembrolizumab, nivolumab, and cemiplimab as anti-PD-1 antibodies, ipilimumab as an anti-CTLA-4 antibody, and atezolizumab, avelumab, and durvalumab as anti-PD-L1 antibodies), and other cancer growth inhibitors including but not limited to tyrosine kinase inhibitors (including but not limited to alectinib, brigatinib, ceritinib, crizotinib, entrectinib, lorlatinib, ALK, I, bosutinib, dasatinib, imatinib, nilotinib, ponatinib, afatinib, dacomitinib, erlotinib, gefitinib, lapatinib, neratinib, osimertinib, vandetanib, gilteritinib, midostaurin, erdafitinib, ruxolitinib, larotrectinib, axitinib, carbozantinib, lenvatinib, pazopanib, regorafenib, sorafenib, sunitinib, dabrafenib, encorafenib, vemurafenib, acalabrutinib, ibrutinib, binimetinib, cobimetinib, trametinib, abemaciclib, palbociclib, or ribociclib), proteasome inhibitors (including but not limited to ortezomib, carfizomib, ixazomib, delanzomib, oprozomib, and marizomib), mTOR inhibitors (including but not limited to everolimus, sirolimus, temsirolimus, everolimus, sirolimus, sirolimus protein-bound, and everolimus), PI3K inhibitors (including but not limited to copanlisib, alpelisib, idelalisib, duvelisib and umbralisib), histone deacetylase inhibitors (including but not limited to vorinostat, romidepsin, panobinostat, and belinostat), and Hedgehog pathway blockers (including but not limited to vismodegib, sonidegib, and glasdegib).

In various embodiments, the methods for treating or inhibiting tumors comprise also administering immune checkpoint inhibitors (including, but not limited to, pembrolizumab, nivolumab, and cemiplimab as anti-PD-1 antibodies, ipilimumab as an anti-CTLA-4 antibody, and atezolizumab, avelumab, and durvalumab as anti-PD-L1 antibodies, and/or radiotherapy.

In another embodiment, the disclosure provides methods for detecting an αvβ8 (+) tumor or immune cell population, comprising administering to a subject suspected of having an αvβ8 (+) tumor or immune cell population an amount of the polypeptide of any embodiment or combination of embodiments disclosed herein effective to detect the tumor in the subject. Such methods can be used, for example, in fluorescence-activated cell sorting (FACS) or in vivo imaging studies of αvβ8 (+) tumor or immune cell populations. Binding of the polypeptide to the αvβ8 (+) tumor or immune cell population can be detected via standard methods.

In another embodiment, the disclosure provides methods for treating or inhibiting tissue fibrosis, comprising administering to a subject in need thereof an amount of the polypeptide, nucleic acid, expression vector, host cell, and/or pharmaceutical composition of any embodiment or combination of embodiments disclosed herein effective to treat or inhibit the tissue fibrosis in the subject. The methods may be used to treat any tissue fibrosis, including but not limited to pulmonary fibrosis (for example, to treat or inhibit chronic obstructive pulmonary disease and asthma, or to treat or inhibit Idiopathic Pulmonary Fibrosis (IPF)), liver fibrosis (for example, liver fibrosis resulting from colorectal carcinoma), and/or renal fibrosis (see, for example, McCarthy, J Cell Sci (2020) 133 (12): jcs239434.

As used herein, “treat” or “treating” means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

As used herein, “inhibit” or “inhibiting” means reducing the amount, rate, and/or rate of increase of what is being inhibited (for example, metastasis or fibrosis).

When the method comprises treating cancer, “treat” or “treating” means accomplishing one or more of the following: (a) reducing the size or volume of tumors and/or metastases in the subject; (b) limiting any increase in the size or volume of tumors and/or metastases in the subject; (c) increasing survival; (d) reducing the severity of symptoms associated with cancer; (e) limiting or preventing development of symptoms associated with cancer; and (f) inhibiting worsening of symptoms associated with cancer.

The subject may be any subject that has a relevant disorder. In one embodiment, the subject is a mammal, including but not limited to humans, dogs, cats, horses, cattle, etc.

As used herein, an “effective” amount refers to an amount of the polypeptide, fusion protein, nucleic acid, expression vector, and/or host cell that is effective for treating the disorder. The polypeptides, fusion proteins nucleic acids, expression vectors, and/or host cells are typically formulated as a pharmaceutical composition, such as those disclosed above, and can be administered via any suitable route, including but not limited to orally, by inhalation spray, ocularly, intravenously, subcutaneously, intraperitoneally, and intravesicularly in dosage unit formulations containing conventional pharmaceutically acceptable carriers, adjuvants, and vehicles.

Any suitable dosage range may be used as determined by attending medical personnel. Dosage regimens can be adjusted to provide the optimum desired response. A suitable dosage range for the polypeptides or fusion proteins may, for instance, be 0.1 ug/kg-100 mg/kg body weight; alternatively, it may be 0.5 ug/kg to 50 mg/kg; 1 ug/kg to 25 mg/kg, or 5 ug/kg to 10 mg/kg body weight. In some embodiments, the recommended dose could be lower than 0.1 mcg/kg, especially if administered locally (such as by intra-tumoral injection). In other embodiments, the recommended dose could be based on weight/m² (i.e. body surface area), and/or it could be administered at a fixed dose (e.g., 0.05-100 mg). The polypeptides, fusion proteins, nucleic acids, expression vectors, and/or host cells can be delivered in a single bolus, or may be administered more than once (e.g., 2, 3, 4, 5, or more times) as determined by an attending physician.

The polypeptides, fusion proteins, nucleic acids, expression vectors, and/or host cells made be administered as the sole therapeutic agent, or may be administered together with (i.e.: combined or separately) one or more other therapeutic agents, including but not limited to tumor resection, chemotherapy, radiation therapy, and immunotherapy (such as checkpoint inhibitors). In various non-limiting embodiments, other agents that may be administered in the methods of the disclosure for treating cancer include angiogenesis inhibitors (including but not limited to axitinib, bevacizumab, cabozantinib, everolimus, lenalidomide, lenvatinib mesylate, pazopanib, ramucirumab, regorafenib, sorafenib, sunitinib, thalidomide, vandetanib, and ziv-aflibercept), immune checkpoint inhibitors (including, but not limited to, pembrolizumab, nivolumab, and cemiplimab as anti-PD-1 antibodies, ipilimumab as an anti-CTLA-4 antibody, and atezolizumab, avelumab, and durvalumab as anti-PD-L1 antibodies), and other cancer growth inhibitors including but not limited to tyrosine kinase inhibitors (including but not limited to alectinib, brigatinib, ceritinib, crizotinib, entrectinib, lorlatinib, ALK, I, bosutinib, dasatinib, imatinib, nilotinib, ponatinib, afatinib, dacomitinib, erlotinib, gefitinib, lapatinib, neratinib, osimertinib, vandetanib, gilteritinib, midostaurin, erdafitinib, ruxolitinib, larotrectinib, axitinib, carbozantinib, lenvatinib, pazopanib, regorafenib, sorafenib, sunitinib, dabrafenib, encorafenib, vemurafenib, acalabrutinib, ibrutinib, binimetinib, cobimetinib, trametinib, abemaciclib, palbociclib, or ribociclib), proteasome inhibitors (including but not limited to ortezomib, carfizomib, ixazomib, delanzomib, oprozomib, and marizomib), mTOR inhibitors (including but not limited to everolimus, sirolimus, temsirolimus, everolimus, sirolimus, sirolimus protein-bound, and everolimus), PI3K inhibitors (including but not limited to copanlisib, alpelisib, idelalisib, duvelisib and umbralisib), histone deacetylase inhibitors (including but not limited to vorinostat, romidepsin, panobinostat, and belinostat), and Hedgehog pathway blockers (including but not limited to vismodegib, sonidegib, and glasdegib).

### Examples

### Abstract

The RGD (Arg-Gly-Asp)-binding integrins αvβ6 and αvβ8 are clinically validated cancer and fibrosis targets of considerable therapeutic importance. Compounds that can discriminate between the two closely related integrin proteins and other RGD integrins, stabilize specific conformational states, and have sufficient stability enabling tissue restricted administration could have considerable therapeutic utility. Existing small molecules and antibody inhibitors do not have all of these properties, and hence there is a need for new approaches. Here a method is described for computationally designing hyperstable RGD-containing miniproteins that are highly selective for a single RGD integrin heterodimer and conformational state, and use this strategy to design inhibitors of αvβ6 and αvβ8 with high selectivity. The αvβ8 inhibitors have picomolar affinities for their target, and >1000-fold selectivity over other RGD integrins. CryoEM structures are within 0.6-0.7 Å root-mean-square deviation (RMSD) to the computational design models, and the αvβ8 inhibitor maintains the constitutively fixed extended-closed αvβ8 conformation.

Binding data was collected on an Octet RED96 (Forte Bio) and processed using the instrument's software. His Tagged and avi-tagged protein binders were immobilized on Ni-NTA and streptavidin sensor tips. The tips were then dipped into wells containing different concentrations of αvβ6 and αvβ8. Association and dissociation steps were recorded for 900 s and 1200 s respectively. Dissociation contestant (kD) was calculated using ForteBio software. An empty sensor with no loaded binding protein was included to discard any non-specific binding of αvβ6 to the octet tip.

### Computational Design Strategy:

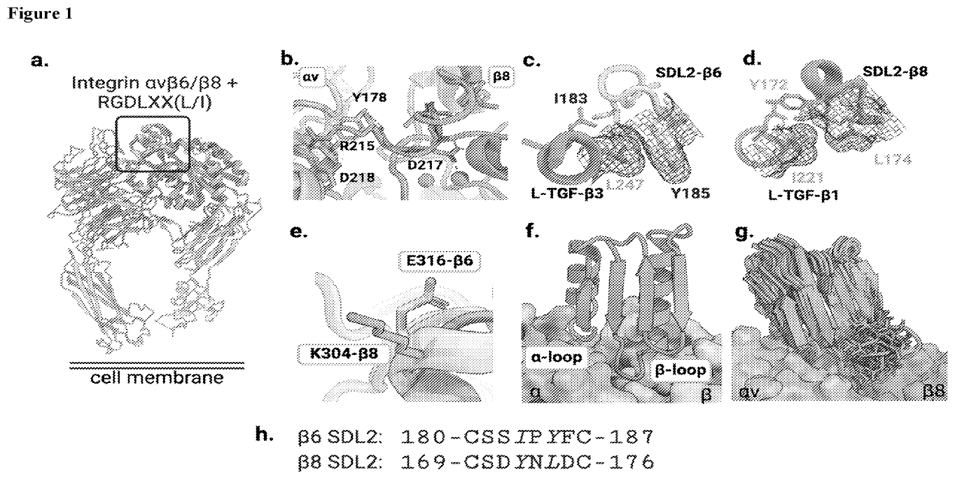

To overcome the limitations of integrin-targeted small molecules and antibodies, a computational approach was developed that generates small (<75 amino acids) hyperstable de novo integrin binding proteins that have high integrin selectivity and specific receptor binding interfaces optimal for treating disease. Integrin αvβ6 and αvβ8 both bind to a RGDLXX(L/I) (SEQ ID NO: 7) motif in the pro-domains of L-TGF-β1 and B3 with low nM affinity (FIG. 1a).^{1,2} As in other structures of RGD-containing peptides bound to integrins, the arginine and aspartate side chains make multiple hydrogen bond and salt-bridge interactions to residues at the interface between the integrin alpha and beta subunits (FIG. 1b). For both αvβ6 and αvβ8, C-terminal to the RGD, the peptide adopts an alpha-helix-like turn with two leucines (or Ile for β8) fitting into a hydrophobic pocket formed by a β6/β8 subunit specificity determining loop 2 (SDL2, FIG. 1c, 1d).¹⁻³ In the unliganded state, SDL2 of αvβ6 is ordered with multiple backbone hydrogen bonds (PDB ID 4UM8), whereas SDL2 of unliganded αvβ8 is mainly flexible.¹⁻³ To engineer selectivity, two main areas on the β subunit were assessed that differ between the two targets: the region that contacts the LXX (L/I) (SEQ ID NO: 8) motif in the L-TGF-β3 peptide (FIG. 1c, 1d) and a charge reversal on the β subunit (FIG. 1e). There are several key differences in the hydrophobic packing pattern of LXX (L/I) (SEQ ID NO: 8) motif and SDL2 of B6 compared to β8 (FIG. 1d, 1d, 1h). Y185 from SDL2 of αvβ6 packs optimally with Leu (LXX (L/I) (SEQ ID NO: 8), L247) of the L-TGF-β3 peptide (PDB ID 4UM9, FIG. 1c), while the equivalent position on the SDL2 of αvβ8 (L174) packs much less tightly with Ile (LXX (L/I) (SEQ ID NO: 8), I221) of L-TGF-β1 (PDB ID 60M2, FIG. 1d). There is also a key charge reversal on the β subunit; β8 contains K304 whereas the equivalent position on β6 is E316 (FIG. 1e). It was hypothesized that minibinders interacting with the Y185/L174 and E316/K304 regions of αvβ6 and αvβ8, respectively, might be able to achieve selectivity between the two proteins.

Small α/β ferredoxin folds (FIG. 1f) were able to scaffold the RGDLXX (L/I) (SEQ ID NO: 7) binding loop without clashing with the integrin while making additional contacts with both α and β subunit (α- and β-loop respectively, FIG. 1f).

To achieve selectivity for the β8 subunit, the β-loop was redesigned to take advantage of the K304 charge reversal on the β subunit (FIG. 1g). We generated 200 models with different lengths and conformations of the β-loop using RosettaRemodel⁴ and the resulting models were superimposed on the L-TGF-β1/αvβ8 complex structure (PDB ID 6OM2) by superposition on the RGD peptide (FIG. 1g). The packing of the L-TGF-β1 LXX (L/I) (SEQ ID NO: 8) motif with SDL2 of αvβ8 integrin is suboptimal (FIG. 1d), it was hypothesized that a minibinder mimicking this interaction would be able to accommodate bulkier residues at these positions, giving additional selectivity. Both the β-loop and LXX (L/I) (SEQ ID NO: 8) motif were redesigned using Rosetta and a total of 9 designs with lowest predicted binding energy were selected following structure prediction using AlphaFold.⁵

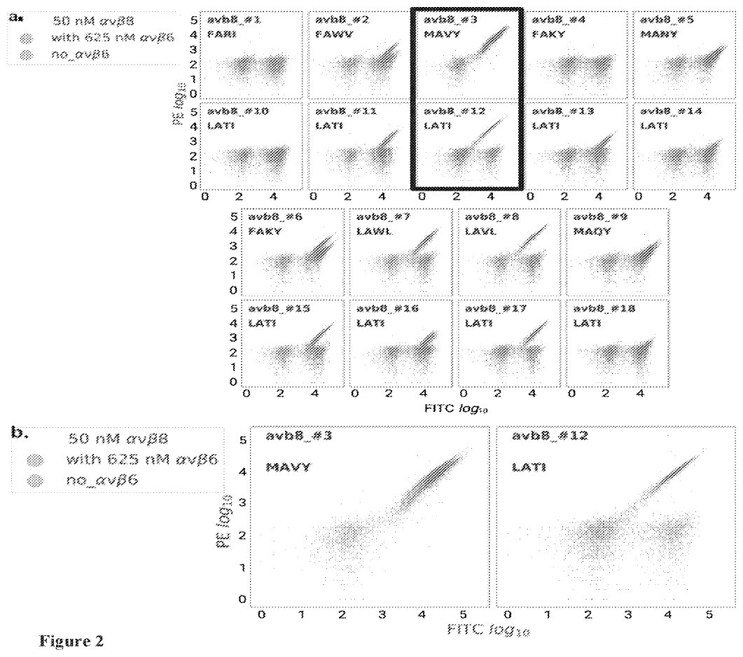

Five out of 9 designs showed preferential binding to αvβ8 integrin with B8_BP_dslf (SEQ ID NO:2) showing the highest affinity and selectivity towards αvβ8 (FIG. 2a). These five designs are: avb8_#2 (SEQ ID NO. 1), avb8_#3 (SEQ ID NO. 3), avb8_#5 (SEQ ID NO. 4), avb8_#6 (SEQ ID NO. 5), and avb8_#9 (SEQ ID NO. 6) (FIG. 2a and Table 2). As shown in FIG. 2a, only these five designs have two populations in the top right quadrant of their flow cytometry panels. avb8_#3 shows the highest binding affinity to αvβ8 in the presence and absence of αvβ6 competition, and the remaining four designs show lower binding affinity to αvβ8 (FIG. 2a). Qualitative αvβ8 binding affinity for these five designs is listed as a column in Table 2. Mutations from the B8_BP_dslf (SEQ ID NO:2) sequence motif MAVY (SEQ ID NO: 14) to LATI (SEQ ID NO: 10) (present in avb8_#12, which corresponds to the L-TGF-β1 peptide sequence) completely abrogated selectivity towards αvβ8 on the yeast surface displayed design (FIG. 2b), indicating these residues are critical for selectivity against αvβ8 vs. αvβ6. B8_BP_dslf is a monomeric and hyperstable protein when expressed in E. coli and binds to human αvβ8 with 1.9 nM affinity, with no appreciable binding to human αvβ6 up to 1 μM (FIG. 3a). In agreement with yeast surface display data, purified avb8_12 with the reversion mutations loses selectivity towards αvβ8 and binds to αvβ6 with a Kd of 1.13 nM (FIG. 3a), confirming the importance of the LXX (L/I) (SEQ ID NO: 8) motif for selectivity. B8_BP_dslf has a MAVY (SEQ ID NO: 14) motif that packs against SDL2 of αvβ8. Each position within the LATI (SEQ ID NO: 10) motif was systematically varied to determine which residue plays a critical role in determining selectivity (FIG. 3b). A single mutation was found containing the LATY (SEQ ID NO: 9) (B8_BP-LATY; SEQ ID NO:3) motif binds to αvβ8 with an affinity of 500 μM with no appreciable binding to αvβ6 at 500 nM concentration (FIG. 3b).

### Selectivity Profiles of B8_BP_Dslf Binder

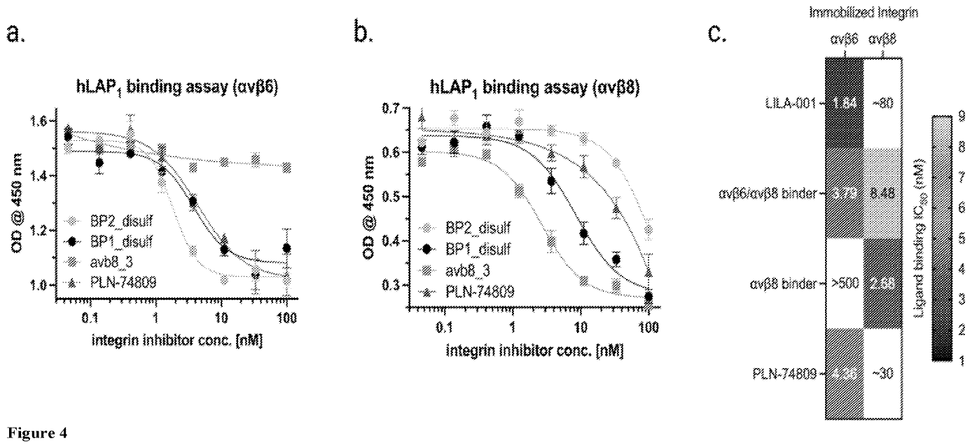

The potency and selectivity of the designed αvβ6 and αvβ8 minibinders were compared to the small-molecule dual αvβ6/αvβ1 inhibitor (PLN-74809) currently in clinical trials as an oral IPF therapy,⁶ by assessing their ability to outcompete binding of hLAP₁, the endogenous ligand of αvβ6 and αvβ8. For αvβ6 integrin, BP2_disulf had the lowest IC₅₀ (1.84 nM), followed by BP1_disulf (dual inhibitor, 3.79 nM), PLN-74809 (4.36 nM), and no significant binding of B8_BP_dslf to αvβ6 integrin was detected (FIG. 4a, 4c). For αvβ8, B8_BP_dslf outcompeted hLAP₁ with the lowest IC₅₀ (2.68 nM), followed in order of potency by BP1_disulf (8.48 nM), PLN-74809, and BP2_disulf (FIG. 4b, 4c). Taken together, these data confirm that B8_BP_dslf has exquisite selectivity and affinity for αvβ8 integrin.

CryoEM Model of αvβ8-B8_BP_Dslf Complex

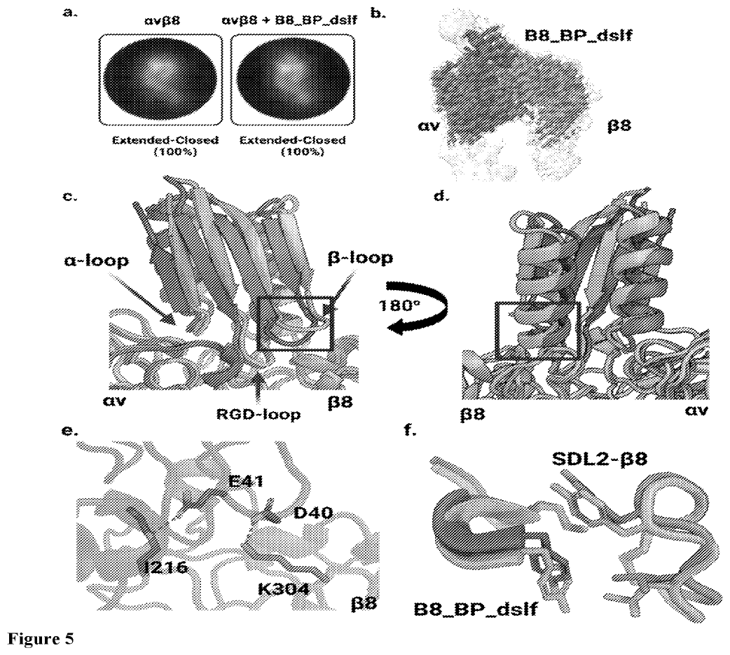

αvβ8 has been shown to bind and activate L-TGF-β while exclusively occupying the extended-closed conformation.³ B8_BP_dslf does not have an effect on the global conformation of αvβ8 and the headpiece remains closed (FIG. 5a).

The cryoEM model of αvβ8-B8_BP_dslf complex is also very close to the computational design model (FIGS. 5b, 5c and 5d, complex RMSD 0.7 Å). In the cryoEM model of the αvβ8-B8_BP_dslf complex, there are 12 interacting pairs of residues between integrin and the minibinder; as in the design model, the α-loop interacts with av, β-loop with B8, and the RGD loop spans the two subunits (FIG. 3e). Y172 of the β8-SDL2 loops bends inward to form a hydrophobic patch similar to the conformation in L-TGF-β-bound structures (FIG. 5f).³ Altering the known binding motif LXX (L/I) (SEQ ID NO: 8) to LATY (SEQ ID NO: 9) was shown to confer selectivity for αvβ8 (FIG. 3b) and the cryoEM structure reveals the molecular basis for this selectivity: Y16 forms stabilizing interactions with A115 of the B8 subunit and interacts with the less bulky L174 in the B8-SDL2 loop (FIG. 5f). The equivalent position in the 6-SDL2 loop, Y185, is too bulky and it is hypothesized that the steric clash would interfere with binding.

### REFERENCES

1. Dong, X., Hudson, N. E., Lu, C. & Springer, T. A. Structural determinants of integrin β-subunit specificity for latent TGF-β. Nat. Struct. Mol. Biol. 21, 1091-1096 (2014).
2. Wang, J., Su, Y., Iacob, R. E., Engen, J. R. & Springer, T. A. General structural features that regulate integrin affinity revealed by atypical αVβ8. Nat. Commun. 10, 5481 (2019).
3. Campbell, M. G. et al. Cryo-EM Reveals Integrin-Mediated TGF-β Activation without Release from Latent TGF-β. Cell 180, 490-501.e16 (2020).
4. Huang, P.-S. et al. RosettaRemodel: a generalized framework for flexible backbone protein design. PLOS One 6, e24109 (2011).
5. Jumper, J. et al. Highly accurate protein structure prediction with AlphaFold. Nature 596, 583-589 (2021).
6. Decaris, M. L. et al. Dual inhibition of αvβ6 and αvβ1 reduces fibrogenesis in lung tissue explants from patients with IPF. Respir. Res. 22, 265 (2021).
7. John, A. E. et al. Translational pharmacology of an inhaled small molecule αvβ6 integrin inhibitor for idiopathic pulmonary fibrosis. Nat. Commun. 11, 4659 (2020).

We claim:

1. An alpha (v) beta (8) integrin (αvβ8)-selective binding polypeptide comprising or consisting of an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from SEQ ID NO:1-6, not including any amino acid insertions, wherein the polypeptide selectively binds to αvβ8, and wherein:
(a) residues 10-12 relative to the reference sequence are RGD
(b) residue 13 relative to the reference sequence is F, M, or L;
(c) residue 16 relative to the reference sequence is Y or V; and
(d) residue 40 relative to the reference sequence is D, E, or P.

2. The polypeptide of claim 1, wherein residue 41 relative to the reference sequence is D, E, Y, or N.

3. The polypeptide of claim 1 or 2, wherein residue 15 relative to the reference sequence is W, V, T, N, K, or Q.

4. The polypeptide of any one of claims 1-3, wherein residue 16 relative to the reference sequence is Y.

5. The polypeptide of any one of claims 1-4, wherein, relative to the reference sequence, one of the following is true:
(a) relative to the reference sequence, residue 13 is F, residue 15 is W, residue 16 is V, residue 40 is D, and residue 41 is Y;
(b) relative to the reference sequence, residue 13 is M, residue 15 is V, residue 16 is Y, residue 40 is D, and residue 41 is E;
(c) relative to the reference sequence, residue 13 is L, residue 15 is T, residue 16 is Y, residue 40 is D, and residue 41 is E;
(d) relative to the reference sequence, residue 13 is M, residue 15 is N, residue 16 is Y, residue 40 is P, and residue 41 is N;
(e) relative to the reference sequence, residue 13 is F, residue 15 is K, residue 16 is Y, residue 40 is E, and residue 41 is E; or
(f) relative to the reference sequence, residue 13 is M, residue 15 is Q, residue 16 is Y, residue 40 is P, and residue 41 is N.

6. The polypeptide of any one of claims 1-4, wherein, relative to the reference sequence, one of the following is true:
(a) relative to the reference sequence, residue 13 is M, residue 15 is V, residue 16 is Y, residue 40 is D, and residue 41 is E; or
(b) relative to the reference sequence, residue 13 is L, residue 15 is T, residue 16 is Y, residue 40 is D, and residue 41 is E.

7. The polypeptide of any one of claims 1-6, wherein residue 13, relative to the reference sequence, is M or L.

8. The polypeptide of any one of claims 1-7, wherein residue 15, relative to the reference sequence, is Tor V.

9. The polypeptide of any one of claims 1-8, wherein residues 13-16, relative to the reference sequence, are LATY (SEQ ID NO:9).

10. The polypeptide of any one of claims 1-8, wherein residues 13-16, relative to the reference sequence, are MAVY (SEQ ID NO:14).

11. The polypeptide of any one of claims 1-6, wherein 1, 2, 3, 4, 5, 6, 7, 8, 9, 10, 11, 12, 13, 14, or all 15 of residues 13-16, 39-42, and 63-69, relative to the reference sequence, are identical to the reference sequence.

12. The polypeptide of any one of claims 1-11, comprising or consisting of an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from SEQ ID NO:2-3, not including any amino acid insertions.

13. The polypeptide of any one of claims 1-12, wherein amino acid changes from the reference protein are conservative amino acid substitutions.

14. The polypeptide of any one of claims 1-12, wherein amino acid substitutions relative to the reference sequence are selected from the amino acid substitutions in Table 2.

15. The polypeptide of any one of claims 1-14, further comprising one or more additional functional domains added at the N-terminus, the C-terminus of the polypeptide, and/or one or more insertion sites selected from residues 28-31, 38, 48, and 50-52 relative to the reference sequence.

16. The polypeptide of any one of claims 1-15, wherein the polypeptide binds to the human αvβ8 integrin with a picomolar affinity (K_{d}) that is at least 1,000-fold higher affinity than the affinity at which the polypeptide binds to any one of another human RGD integrins selected from the group consisting of αvβ1, αvβ3, αvβ5, αvβ6, α5β1, α8β1, and αIIbβ3.

17. A nucleic acid encoding the polypeptide of any one of claims 1-15.

18. An expression vector comprising the nucleic acid of claim 17 operatively linked to a control sequence.

19. A host cell comprising the nucleic acid of claim 17 and/or the expression vector of claim 18.

20. A recombinant cell expressing the polypeptide of any one of claims 1-16.

21. A pharmaceutical composition comprising:
(a) the polypeptide, nucleic acid, expression vector, host cell, or recombinant cell of any one of claims 1-20; and
(b) a pharmaceutically acceptable carrier.

22. A method for treating or inhibiting an αvβ8 (+) tumor, comprising administering to a subject in need thereof an amount of the polypeptide, nucleic acid, expression vector, host cell, and/or pharmaceutical composition of any one of claims 1-21 effective to treat or inhibit the tumor in the subject.

23. The method of claim 22, wherein the method further comprises administering to the subject one or more other therapeutic agents selected from the group consisting of radiation therapy, an angiogenesis inhibitor, a protein kinase inhibitor, a proteasome inhibitor, an immune checkpoint inhibitor, an mTOR inhibitor, a PI3K inhibitor, a histone deacetylase inhibitor, and a Hedgehog pathway blocker.

24. The method of claim 23, wherein the angiogenesis inhibitors is selected from the group consisting of axitinib, bevacizumab, cabozantinib, everolimus, lenalidomide, lenvatinib mesylate, pazopanib, ramucirumab, regorafenib, sorafenib, sunitinib, thalidomide, vandetanib, and ziv-aflibercept; the immune checkpoint inhibitor is selected from the group consisting of pembrolizumab, nivolumab, and cemiplimab as anti-PD-1 antibodies, ipilimumab as an anti-CTLA-4 antibody, and atezolizumab, avelumab, and durvalumab as anti-PD-L1 antibodies), the tyrosine kinase inhibitor is selected from the group consisting of alectinib, brigatinib, ceritinib, crizotinib, entrectinib, lorlatinib, ALK, I, bosutinib, dasatinib, imatinib, nilotinib, ponatinib, afatinib, dacomitinib, erlotinib, gefitinib, lapatinib, neratinib, osimertinib, vandetanib, gilteritinib, midostaurin, erdafitinib, ruxolitinib, larotrectinib, axitinib, carbozantinib, lenvatinib, pazopanib, regorafenib, sorafenib, sunitinib, dabrafenib, encorafenib, vemurafenib, acalabrutinib, ibrutinib, binimetinib, cobimetinib, trametinib, abemaciclib, palbociclib, or ribociclib, the proteasome inhibitor is selected from the group consisting of ortezomib, carfizomib, ixazomib, delanzomib, oprozomib, and marizomib; the mTOR inhibitor is selected from the group consisting of everolimus, sirolimus, temsirolimus, everolimus, sirolimus, sirolimus protein-bound, and everolimus; the PI3K inhibitor is selected from the group consisting of copanlisib, alpelisib, idelalisib, duvelisib and umbralisib; the histone deacetylase inhibitor is selected from the group consisting of vorinostat, romidepsin, panobinostat, and belinostat; and/or the Hedgehog pathway blocker is selected from the group consisting of vismodegib, sonidegib, and glasdegib.

25. The method of any one of claims 22-24, wherein the tumor is selected from the group consisting of a colorectal tumor, a glioblastoma, and a melanoma

26. A method for detecting an αvβ8 (+) tumor or immune cell population, comprising administering to a subject suspected of having an αvβ8 (+) tumor or immune cell population an amount of the polypeptide of any one of claims 1-16 effective to detect the tumor in the subject.

27. A method for treating or inhibiting tissue fibrosis, comprising administering to a subject in need thereof an amount of the polypeptide, nucleic acid, expression vector, host cell, and/or pharmaceutical composition of any one of claims 1-21 effective to treat or inhibit the tissue fibrosis in the subject.

28. The method of claim 27, wherein the tissue fibrosis is selected from the group consisting of pulmonary fibrosis, liver fibrosis, and renal fibrosis.

29. The method of claim 28, wherein the tissue fibrosis comprises Idiopathic Pulmonary Fibrosis (IPF).

30. Use of the polypeptide, nucleic acid, expression vector, host cell, and/or pharmaceutical composition of any one of claims 1-21 for treating or inhibiting an αvβ8 (+) tumor in a subject.

31. The use of claim 30, wherein the tumor is selected from the group consisting of a colorectal tumor, a glioblastoma, and a melanoma.

32. The use of claim 30 or 31, wherein the use further comprise use of one or more other therapeutic agents selected from the group consisting of radiation therapy, angiogenesis inhibitors, immune checkpoint inhibitors, mTOR inhibitors, PI3K inhibitors, histone deacetylase inhibitors, and Hedgehog pathway blockers

33. The use of claim 32, wherein the angiogenesis inhibitor is selected from the group consisting of axitinib, bevacizumab, cabozantinib, everolimus, lenalidomide, lenvatinib mesylate, pazopanib, ramucirumab, regorafenib, sorafenib, sunitinib, thalidomide, vandetanib, and ziv-aflibercept; the immune checkpoint inhibitor is selected from the group consisting of pembrolizumab, nivolumab, and cemiplimab as anti-PD-1 antibodies, ipilimumab as an anti-CTLA-4 antibody, and atezolizumab, avelumab, and durvalumab as anti-PD-L1 antibodies), the tyrosine kinase inhibitor is selected from the group consisting of alectinib, brigatinib, ceritinib, crizotinib, entrectinib, lorlatinib, ALK, I, bosutinib, dasatinib, imatinib, nilotinib, ponatinib, afatinib, dacomitinib, erlotinib, gefitinib, lapatinib, neratinib, osimertinib, vandetanib, gilteritinib, midostaurin, erdafitinib, ruxolitinib, larotrectinib, axitinib, carbozantinib, lenvatinib, pazopanib, regorafenib, sorafenib, sunitinib, dabrafenib, encorafenib, vemurafenib, acalabrutinib, ibrutinib, binimetinib, cobimetinib, trametinib, abemaciclib, palbociclib, or ribociclib, the proteasome inhibitor is selected from the group consisting of ortezomib, carfizomib, ixazomib, delanzomib, oprozomib, and marizomib; the mTOR inhibitor is selected from the group consisting of everolimus, sirolimus, temsirolimus, everolimus, sirolimus, sirolimus protein-bound, and everolimus; the PI3K inhibitor is selected from the group consisting of copanlisib, alpelisib, idelalisib, duvelisib and umbralisib; the histone deacetylase inhibitor is selected from the group consisting of vorinostat, romidepsin, panobinostat, and belinostat; and/or the Hedgehog pathway blocker is selected from the group consisting of vismodegib, sonidegib, and glasdegib

34. Use of the polypeptide of any one of claims 1-16 for detecting an αvβ8 (+) tumor or immune cell population.

35. Use of the polypeptide, nucleic acid, expression vector, host cell, and/or pharmaceutical composition of any one of claims 1-21 for treating or inhibiting tissue fibrosis.

36. The use of claim 35, wherein the tissue fibrosis is selected from the group consisting of pulmonary fibrosis, liver fibrosis, and renal fibrosis.

37. The use of claim 36, wherein the tissue fibrosis comprises Idiopathic Pulmonary Fibrosis (IPF).
